(19) United States
(12) Patent Application Publication
(10) Pub. No.: US 2024/0294865 A1
(43) Pub. Date: Sep. 5, 2024

(54) PRODUCTION OF SINGLE CELL PROTEIN BY GAS FERMENTATION

(71) Applicant: SUPERBREWED FOOD, INC., New Castle, DE (US)

(72) Inventors: Pradeep MUNASINGHE, Wilmington, DE (US); Biniam MARU, Bear, DE (US); Hadar GILARY, Magal (IL); Bryan P. TRACY, Wilmington, DE (US); Alon KARPOL, Tel Mond (IL)

(21) Appl. No.: 18/696,393

(22) PCT Filed: Sep. 25, 2022
(86) PCT No.: PCT/IB2022/059061

§ 371 Date: Mar. 28, 2024

Related U.S. Application Data
(60) Provisional application No. 63/249,310, filed on Sep. 28, 2021.

(51) Int. Cl. C12N 1/20; C12R 1/145

(52) U.S. Cl. CPC C12N 1/20; C12N 2500/24; C12N 2500/38; C12N 2500/74; C12R 2001/145

(57) ABSTRACT

Provided is a method for the production of single cell protein (SCP) comprising anaerobic fermentation of Clostridia bacterium on a fermentation medium comprising a gas, wherein said gas provides a source of energy and carbon for said bacterium. Further provided is a single cell protein produced by the method as disclosed herein.

### CROSS-REFERENCE TO RELATED APPLICATION

The present application gains priority from U.S. Provisional Application No. 63/249,310 filed Sep. 28, 2021 which is incorporated by reference as if fully set-forth herein.

### FIELD OF THE INVENTION

The present invention relates to production of single cell protein by gas fermentation, and in at least some embodiments to production of single cell protein by anaerobic fermentation of Clostridia bacterial in a fermentation medium comprising an inorganic gaseous feedstock.

### BACKGROUND OF THE INVENTION

Protein is an essential part of any diet. Increasing world population increases the demand for protein. Vegetable proteins are less attractive sources, since they are relatively poor in some essential amino acids, e.g. lysine, methionine, tryptophan and tyrosine. Animal sources provide the full range of essential amino acids needed by humans in sufficient quantities. The way in which protein is produced has significant impacts on both the environment and human health.

Single-cell protein provides a source of high-value proteins. There is a need for improved methods for the preparation of single-cell protein.

### SUMMARY OF THE INVENTION

According to an aspect of some embodiments of the present invention, there is provided a method for the production of single cell protein (SCP) comprising anaerobic fermentation of Clostridia bacterium on a fermentation medium comprising an inorganic gaseous feedstock.

According to a further aspect of some embodiments of the present invention, there is provided a single cell protein (SCP) produced by the method as disclosed herein.

### DETAILED DESCRIPTION OF THE INVENTION

The present invention, in at least some embodiments thereof, provides an improved method for the production of single cell protein (SCP) comprising anaerobic fermentation of Clostridia bacterium on a fermentation medium comprising an inorganic gaseous feedstock.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise stated, the term “about” as used herein is intended to mean ±10%.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

As used herein, the term “anaerobic fermentation” refers to fermentation of an organic or inorganic carbon to a product such an organic acid, gas and/or alcohol in an anaerobic environment.

As used herein, the term “anaerobic environment” refers to an environment having less than about 100 mM of oxygen.

As used herein, the term “non genetically engineered” with respect to a bacterium refers to a bacterium in which DNA has not been modified using genetic engineering.

As used herein, the term “syngas” refers to a mixture of carbon monoxide and hydrogen, optionally further comprising carbon dioxide.

As used herein, the term “butyrate producer” refers to a bacterium having the necessary genes and pathways for the formation of at least 0.1 g/L butyrate, the anion of butyric acid (butanoic acid), from acetyl-CoA. Such genes can include, but are not limited to, thiolase (also known as acetyl-CoA acetyltransferase), 3-hydroxybutyl-CoA dehydrogenase, crotonase (also known as enoyl-CoA hydratase), butyryl-CoA dehydrogenase, trans-2-enoyl-CoA reductase, phosphate butyryltransferase, and butyrate kinase.

As used herein, the terms “CO₂-utilizing microorganism” and “CO-utilizing microorganism” refer to microorganisms having the ability to fix organic carbon dioxide and carbon monoxide, respectively, to form organic carbon compounds, while the terms “non-CO₂-utilizing microorganism” and “non-CO-utilizing microorganism” refer to microorganisms which are devoid of the aforementioned ability.

As used herein, the term “continuous mode” with regard to a fermentation process refers to a process in which a fresh culture medium is provided, and end products are removed throughout the process.

As used herein, the term “batch mode” with regard to a fermentation process refers to a process in which an initial culture medium is provided, and fermentation is allowed to proceed to completion with no further input of medium or removal of products.

As used herein, the term “autotrophic conditions” refers to conductions under with a bacterium is capable of forming nutritive organic materials from inorganic materials.

According to an aspect of some embodiments of the present invention, there is provided a method for the production of single cell protein (SCP) comprising anaerobic fermentation of Clostridia bacterium on a fermentation medium comprising an inorganic gaseous feedstock.

According to some embodiments, said Clostridia bacterium comprises a fast-growing strain, such as one having a dilution rate >2 1/day.

According to some embodiments, said inorganic gaseous feedstock provides a sole source of energy and carbon for said bacterium.

According to some embodiments, said inorganic gaseous feedstock is selected from the group consisting of carbon monoxide; a mixture of hydrogen with carbon monoxide or with carbon dioxide; and a mixture of carbon dioxide with hydrogen or with carbon monoxide.

According to some embodiments, said Clostridia bacterium is a CO₂-utilizing microorganism. According to some embodiments, said Clostridia bacterium is a CO-utilizing microorganism.

According to some embodiments, said inorganic gaseous feedstock comprises syngas. According to some such embodiments, said syngas comprises up to about 80% hydrogen (such as about 0%, about 10%, about 20%, about 30%, about 40%, about 50%, about 60%, about 70% or 80%), up to about 90% carbon monoxide such as about 0%, about 10%, about 20%, about 30%, about 40%, about 50%, about 60%, about 70%, about 80% or 90%), and up to about 80% carbon dioxide (such as about 0%, about 10%, about 20%, about 30%, about 40%, about 50%, about 60%, about 70% or 80%). According to some such embodiments, said syngas comprises about 25% hydrogen, about 70% carbon monoxide and about 5% carbon dioxide.

According to some embodiments, said Clostridia bacterium is an acetogen.

According to some such embodiments, fixation of the inorganic gaseous feedstock by said acetogenic bacterium can be accomplished through the reductive acetyl-coenzyme A pathway, also known as the Wood-Ljungdahl pathway. Alternatively or additionally, fixation of inorganic feedstock is accomplished by at least one carbon fixation pathway selected from the group consisting of the reductive pentose phosphate cycle; the reductive TCA cycle; the dicarboxylate-4-hydroxybutyrate Cycle; the 3-Hydroxypropionate-4-hydroxybutyrate cycle; the 3-Hydroxypropionate bicycle; the reductive acetyl-CoA pathway (acetogens); the reductive acetyl-CoA pathway (methanogens); the glycine synthase pathway (glycine reductase); the glycine synthase pathway (serine hydroxymethytransferase); the glyoxylate synthetase pathway and combinations thereof.

According to some embodiments, said Clostridia bacterium is a butyrate producer.

According to some embodiments, said Clostridia bacterium is isolated from a microbiome of an animal, such as a human, a horse, a rabbit, a sheep, a gorilla, a cow and a chicken. According to some such embodiments, said Clostridia bacterium comprises Eubacterium callendari.

According to some embodiments, at least a portion of said fermentation is conducted in continuous mode. According to some such embodiments, an initial portion of said fermentation is conducted in batch mode and a subsequent portion is conducted in continuous mode.

According to some embodiments, at least a portion of said fermentation is conducted under autotrophic conditions.

According to some embodiments, said fermentation medium further comprises at least one selected from the group consisting of yeast extract, ferrous sulfate heptahydrate, and at least one vitamin.

According to some such embodiments, said at least one vitamin is selected from the group consisting of biotin, thioctic acid, calcium pantothenate, nicotinic acid, riboflavin, thiamine hydrochloride and pyridoxine hydrochloride.

According to some such embodiments, said vitamins comprise Wolfe's vitamin solution.

According to an aspect of some embodiments of the present invention, there is provided a single cell protein (SCP) produced by the method as disclosed herein.

According to some embodiments, the SCP has a crude protein concentration of greater than 80wt % and an essential amino acid concentration of greater than 30wt %.

### EXAMPLES

Example 1: SCP production by gas fermentation with Eubacterium callanderi (US#2) strain

A continuous cell recycle fermentation was carried out in a 3L fermenter with Eubacterium callanderi strain (US#2) under autotrophic conditions. The culture was initially kept on batch condition for 65 h, before starting the continuous cell recycle fermentation. GE hollow fiber membrane (0.1 um, 0.9 ft 2) was used in cell recycle fermentation with 1.7 mL/min permeate and feed rate, at the beginning of the process. The optical density (OD) of the culture reached 34 (7.3 g/Kg cell titer) after 138 h of fermentation with 8.9 g/L of acetate and 0.12 g/L of butyrate. Cell harvesting (1 mL/min) was started at 165 h. The highest acetate concentration of 11 g/L was observed just before starting the harvest. Cell recycle was continued for 240 h before shutting the fermenter down.

The following 3 L fermentation conditions were used:

Clj std media with 1 g/L of yeast extract, 0.1 g/L of FeSO4.7H20 and 20 mL/L of Wolfe's vitamin was used in the 3L fermenter, and the feed carboys used in cell recycle. Syngas mixer of 25% H_{2, 70}% CO, and 5% CO₂ was sparged through the fermenter and the headspace was pressurized to 15-18 psig using a back-pressure regulator. pH was maintained at 6.5-6.7 and NH₄OH (8M) was used as the base control. 10% of actively growing inoculum (inoculation OD of 1.5-2.0) was used in 3L inoculation. The inoculum was obtained from seed culture which was grown in serum bottles under a syngas mixture of (20% H_{2, 5}% Ar, 10% N₂, 55% CO and 10% CO₂). The agitation of the fermenter was increased from 350 rpm (in batch 1.1L volume) to 700 rpm (cell recycle at 2.5L working volume). Samples were harvested at 134 h and 190 h for cell dry weight and crude protein analysis.

Summary of results

| | | | Cell | OD | Crude |
|---|---|---|---|---|---|
| | | Acetate | Titer | Factor | protein |
| Sample | OD | (g/L) | (g/Kg) | (g/Kg · OD) | (%) |
| 134 h Harvest | 32.7 | 9.5 | 7.28 | 0.22 | 82% |
| 190 h Harvest | 11.8 | 4.1 | 2.93 | 0.25 | 81% |

Exemplary amino acid compositions of SCP produced according to the method of the present invention (from Example 1)

Amino acid percentage of total amino acid (wt/wt) (based on mg amino acid/mg dry sample)

| | | % AA of total AA (wt/wt) | |
|---|---|---|---|
| | | AL200526 | AL200526 |
| | Amino acid | 134 hr | 190 hr |
| Essential | Histidine | 1.15% | 1.99% |
| | Isoleucine | 6.27% | 6.28% |
| | Leucine | 7.50% | 7.60% |
| | Lysine | 7.29% | 7.41% |
| | Methionine | 1.85% | 1.90% |
| | Phenylalanine | 3.78% | 4.14% |
| | Threonine | 4.98% | 4.82% |
| | Tryptophan | — | — |
| | Valine | 5.31% | 5.51% |
| Non-essential | Alanine | 8.19% | 7.89% |
| | Arginine | 6.71% | 5.37% |
| | Aspartic | 11.93% | 11.08% |
| | acid/asparagine | | |
| | Glutamic | 18.04% | 18.31% |
| | acid/glutamine | | |
| | Cysteine | — | — |
| | Glycine | 4.51% | 4.25% |
| | Proline | 5.60% | 6.65% |
| | Serine | 4.06% | 3.82% |
| | Tyrosine | 2.83% | 2.97% |
| | | 100.00% | 100.00% |

Estimated corrected weight % (mg amino acid/mg dry sample)

| | Amino acid | Average | |
|---|---|---|---|
| Essential | Histidine | 1.57% | |
| | Isoleucine | 6.27% | |
| | Leucine | 7.55% | |
| | Lysine | 7.35% | |
| | Methionine | 1.88% | |
| | Phenylalanine | 3.96% | |
| | Threonine | 4.90% | |
| | Tryptophan | — | |
| | Valine | 5.41% | Total essential amino acids: 38.9% |
| Non-essential | Alanine | 8.04% | |
| | Arginine | 6.04% | |
| | Aspartic | 11.51% | |
| | acid/asparagine | | |
| | Glutamic | 18.18% | |
| | acid/glutamine | | |
| | Cysteine | — | |
| | Glycine | 4.38% | |
| | Proline | 6.13% | |
| | Serine | 3.94% | |
| | Tyrosine | 2.90% | Total non-essential amino acids: 61.11% |
| | | 100.00% | |

1. A method for the production of single cell protein (SCP) comprising anaerobic fermentation of Clostridia bacterium on a fermentation medium comprising an inorganic gaseous feedstock.

2. The method of claim 1, wherein said inorganic gaseous feedstock is selected from the group consisting of carbon monoxide; a mixture of hydrogen with carbon monoxide or with carbon dioxide; and a mixture of carbon dioxide with hydrogen or with carbon monoxide.

3. The method of claim 1, wherein said inorganic gaseous feedstock comprises syngas.

4. The method of claim 1, wherein said Clostridia bacterium is an acetogen.

5. The method of claim 1, wherein said Clostridia bacterium is a butyrate producer.

6. The method of claim 1, wherein said Clostridia bacterium is isolated from a microbiome of an animal.

7. The method of claim 1, wherein said animal is of selected from the group consisting of a human, a horse, a rabbit, a sheep, a gorilla, a cow, and a chicken.

8. The method of claim 8, wherein said animal is a grass-eating animal.

9. The method of claim 1, wherein said Clostridia bacterium comprises Eubacterium callendari.

10. The method of claim 1, wherein at least a portion of said fermentation is conducted in continuous mode.

11. The method of claim 11, wherein an initial portion of said fermentation is conducted in batch mode and a subsequent portion is conducted in continuous mode.

12. The method of claim 1, wherein at least a portion of said fermentation is conducted under autotrophic conditions.

13. The method of claim 4, wherein said syngas comprises up to about 80% hydrogen, up to about 90% carbon monoxide, and up to about 80% carbon dioxide.

14. The method of claim 14, wherein said syngas comprises about 25% hydrogen, about 70% carbon monoxide and about 5% carbon dioxide.

15. The method of claim 1, wherein said fermentation medium further comprises at least one selected from the group consisting of yeast extract, ferrous sulfate heptahydrate, and at least one vitamin.

16. The method of claim 16, wherein said at least one vitamin is selected from the group consisting of biotin, thioctic acid, calcium pantothenate, nicotinic acid, riboflavin, thiamine hydrochloride and pyridoxine hydrochloride.

17. A single cell protein (SCP) produced by the method of claim 1.

18. The SCP of claim 18, comprising a crude protein concentration of greater than 80 wt % and an essential amino acid concentration of greater than 30 wt %.
